(19) United States
(12) Patent Application Publication
(10) Pub. No.: US 2019/0343746 A1
(43) Pub. Date: Nov. 14, 2019

(54) COSMETIC COMPOSITION INCLUDING GAMMA-PGA AS ACTIVE INGREDIENT

(71) Applicant: Vedan Biotechnology Corporation, Taichung City (TW)

(72) Inventors: Shih-An Yang, Taichung City (TW); Chin-Feng Chan, Taichung City (TW)

(21) Appl. No.: 16/408,391

(22) Filed: May 9, 2019

(30) Foreign Application Priority Data
May 10, 2018 (TW) 107115915

(51) Int. Cl. A61K 8/44; A61Q 19/08; A61Q 19/00; A61K 8/65; A61K 8/04

(52) U.S. Cl. CPC A61K 8/44; A61Q 19/08; A61K 8/042; A61K 8/65; A61Q 19/007

(57) ABSTRACT

A cosmetic composition including γ-polyglutamic acid or a salt thereof as active ingredients is made of from 0.01% to 5% by weight of γ-polyglutamic acid (γ-PGA) or a salt thereof and from 0.02% to 8% by weight of chlorella growth factor (CGF) based on a total weight of the cosmetic composition. The average molecular weight of the γ-polyglutamic acid or the salt thereof is between 1×10⁶ Da and 3×10⁶ Da.

### CROSS REFERENCE OF RELATED APPLICATION

The present application claims priority of Taiwanese Patent Application No. 107115915, filed on May 10, 2019, which is incorporated herein by reference in its entirety.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates generally to a cosmetic composition, and more particularly to a cosmetic composition including γ-polyglutamic acid as an active ingredient.

### Description of Related Arts

Proper moisturizing and nutrition are essential for the health and beauty of human skin and human hair. Excessive dryness due to low humidity often damages skin and hair. In the winter, low temperature and dry air are the main cause of dry skin and hair, which in turn deteriorates the health of the skin and even hardens or damages the epidermis and generates net charges in the hair. To avoid drying of skin, hair and nails, cosmetics such as skin essences, hand, foot and body lotions, bath soaps, hair gels, shampoos, mousses, and other personal care kits often contain certain moisturizers for the skin and hair. As a result, the moisture in the skin and hair may be retained by using the moisturizers to maintain the quality of the skin and hair.

There are many types of organic moisturizers used in various commercial cosmetic and hygiene products. However, the water absorption capacity, safety requirements, and long-term stability of these moisturizers greatly limit the types of moisturizers used in practical applications. Good moisturizers need to have high moisture retention capacity and reduce water loss due to evaporation from the skin and hair. Conventional moisturizers used in the cosmetic industry include glycerin, diglycerol, sorbitol, sodium lactate, propylene glycol, and amino acids. For sodium lactate, although it has better moisture retention capacity, the application of the sodium lactate is very limited and its use amount is also low since it is difficult to be emulsified in the final product. Polyols have a better moisturizing effect, but they are less effective in cosmetic products. Hyaluronic acid (HA), collagen and squalane have good moisture retention capacity, but they are less effective in reducing the water evaporation rate from the skin and are too viscous when applied on the skin. Furthermore, other moisturizers, such as elastin, glucosamine, polyaspartic acid (see JP 61-033107), placenta, chondroitin, aloe extract, and amino acid ester (see JP 10-251402), are also used in commercial cosmetics and personal care ingredients. However, the cosmetic manufacturers still put a lot of effort into developing better moisturizers.

Polyols such as 1,3-butanediol, ethylene glycol, propylene glycol, and polyethylene glycol have the moisturizing ability and are capable of inhibiting the growth of some microorganisms, improving the miscibility and viscosity of the fluid, and enhancing chemical or physical stability of other ingredients in the cosmetics and personal care products. The cosmetics and personal care products may include nourishing essences, skin creams, skin and body lotions, gels, shampoos, conditioners, anti-drying treatments, hair nourishing liquids, bathing and moisturizing cream, and so forth. Despite the benefits derived from polyols, skincare and hair care cosmetics with the above polyols often let the user feel discomfort or give the hair a slightly dry feel when the user's skin or hair is applied with the cosmetics with the polyols. Thus, the user may think that the cosmetics with the polyols are worse than the cosmetics without the polyols. Furthermore, for facial or hair cleansers containing polyols, the residue of polyols often remains on the skin or hair of the users, and sometimes makes the user feel uncomfortable, and thereby reducing their desire to use these products.

Hyaluronic acid (HA) has excellent water absorption and moisture retention capacity. Specifically, HA is a non-toxic and biocompatible natural biopolymer, and has been used in most high quality cosmetics because of its ability to retain the moisture in the skin and hair. The disadvantage of using HA is its high price and limited source of access. Despite its excellent moisturizing ability to avoid excessive drying of the skin, the extremely high price of HA will result in the relatively high price of the final cosmetic formulations for the final product. The recent BSE viral protein (prion) and the prevalence of avian influenza in Asia have raised serious concerns about the safety of HA. Therefore, although squalane has the advantage of moistening the surface of the skin by avoiding evaporation of water from the surface of the skin, its oily nature makes the user's skin feel greasy. Also, commercially available collagen is often extracted from animals, which means that the collagen may likely be contaminated by BSE (i.e. bovine spongiform encephalopathy) viral proteins or avian influenza viruses.

### SUMMARY OF THE PRESENT INVENTION

In light of the above, a cosmetic composition including γ-polyglutamic acid as an active ingredient is disclosed according to embodiments of the present invention, which can overcome the drawbacks in conventional techniques.

According to one embodiment of the present invention, a cosmetic composition including γ-polyglutamic acid as an active ingredient is disclosed. The cosmetic composition includes γ-polyglutamic acid or its salt and chlorella growth factor. The concentration of the γ-polyglutamic acid (γ-PGA) or the salt thereof is from 0.01 wt. % to 5 wt. % based on the total weight of the cosmetic composition, and an average molecular weight of the γ-polyglutamic acid or the salt thereof is between 1×10⁶ Da and 3×10⁶ Da. The concentration of the chlorella growth factor (CGF) is from 0.02 wt. % to 8 wt. % based on the total weight of the cosmetic composition.

According to one embodiment of the invention, the cosmetic composition further includes an additive with a concentration between 7.66 wt. % and 9.85 wt. %, and the additive is selected from the group consisting of 1,3-butanediol, hydroxyethyl cellulose, sodium glutamate, phenoxyethanol, ethylhexyl glycerol, and imidazolidinyl urea.

According to one embodiment of the present invention, the cosmetic composition further includes γ-polyglutamate hydrogel.

According to one embodiment of the present invention, the average molecular weight of the γ-polyglutamate hydrogel is between 15×10⁶ Da and 200×10⁶ Da.

According to one embodiment of the invention, the γ-polyglutamate hydrogel has a cross-linked structure.

According to one embodiment of the invention, the cosmetic composition is capable of reducing skin wrinkles of a user.

According to one embodiment of the invention, the cosmetic composition is capable of increasing the collagen content of a user's skin.

According to one embodiment of the invention, the cosmetic composition is capable of increasing skin elasticity of a user.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

For a more complete understanding of the embodiments of the present invention and their advantage, reference is now made to the following description, taken in conjunction with accompanying drawings, in which:

FIG. 1 shows the amino acid composition of proteins of chlorella growth factor (CGF) according to one embodiment of the present invention.

FIG. 2 is a flow chart showing a procedure for culturing cells with a medium containing a cosmetic composition according to one embodiment of the present invention.

FIG. 3 shows the results of the effect of cosmetic ingredients (γ-PGA hydrogel) on promoting the growth of 3T3 cells according to one embodiment of the present invention.

FIG. 4 shows the results of the effect of cosmetic ingredients (CGF) on promoting the growth of 3T3 cells according to one embodiment of the present invention.

FIG. 5 shows the results of the effect of a cosmetic composition (2D) on promoting the growth of 3T3 cells according to one embodiment of the present invention.

FIG. 6 shows the results of the effect of a cosmetic composition (2D) on promoting the growth of HaCaT cells according to one embodiment of the present invention.

FIG. 7 shows the results of the effect of a cosmetic composition (3D&2D) on promoting the growth of 3T3 cells according to one embodiment of the present invention.

FIG. 8 shows the results of the effect of a cosmetic composition (3D&2D) on promoting the growth of HaCaT cells according to one embodiment of the present invention.

FIG. 9 shows the test results of ex vivo moisture retaining ability of different cosmetic compositions (γ-PGA hydrogel) according to one embodiment of the present invention.

FIGS. 10 and 11 respectively show the effect of different cosmetic compositions (γ-PGA hydrogel) on the moisture content of skin according to one embodiment of the present invention.

FIG. 12 shows the effect of different cosmetic compositions (γ-PGA hydrogel) on skin water loss according to one embodiment of the present invention.

FIGS. 13 and 14 respectively show the results of UV-induced damage test for different cosmetic compositions according to one embodiment of the present invention.

FIG. 15 shows the results of cell wound healing test for a comparative cosmetic composition according to one embodiment of the present invention.

FIG. 16 shows the results of cell wound healing test for a cosmetic composition according to one embodiment of the present invention.

FIGS. 17 and 18 respectively show the results of skin irritation tests for different cosmetic compositions according to one embodiment of the present invention.

FIG. 19 shows the results of the effect of cosmetic compositions (left face: item 6; right face: item 7) on skin spots, wrinkles, texture, elasticity, collagen content, moisture content and transepidermal water loss according to one embodiment of the present invention.

FIG. 20 shows the results of the effect of cosmetic compositions on skin elasticity and moisture content of skin according to one embodiment of the present invention.

FIG. 21 shows the results of the effect of cosmetic compositions (experimental group and control groups) on skin wrinkles according to one embodiment of the present invention.

FIG. 22 shows the results of the effect of cosmetic compositions (experimental group and control groups) on skin collagen content according to one embodiment of the present invention.

FIG. 23 shows the results of the effect of cosmetic compositions (experimental group and control groups) on skin elasticity according to one embodiment of the present invention.

### DETAILED DESCRIPTION OF THE PREFERRED EMBODIMENT

The invention will be described more fully hereinafter with reference to the accompanying drawings, in which example embodiments of the invention are shown.

This invention may, however, be embodied in many different forms and should not be construed as limited to the example embodiments set forth herein. Rather, the disclosed embodiments are provided so that this disclosure will be thorough and complete, and will fully convey the scope of the invention to those skilled in the art. In the drawings, the size and relative sizes of components and regions may be exaggerated for clarity unless express so defined herein.

The terminology used herein is for describing particular embodiments only and is not intended to be limiting. As used herein, the singular terms “a”, “an,” and “the” may be intended to include the plural forms as well, unless the context clearly indicates otherwise. The terms “comprises,” “comprising,” “includes” and/or “including” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

According to one embodiment of the present invention, a cosmetic composition including γ-polyglutamic acid as an active ingredient is disclosed. The cosmetic composition includes γ-polyglutamic acid or its salt and chlorella growth factor, and may include additional additives. The concentration of the γ-polyglutamic acid (γ-PGA) or the salt thereof is from 0.01 wt. % to 5 wt. % based on the total weight of the cosmetic composition, and an average molecular weight of the γ-polyglutamic acid or the salt thereof is between 1×10⁶ Da and 3×10⁶ Da. The concentration of the chlorella growth factor (CGF) is from 0.02 wt. % to 8 wt. % based on the total weight of the cosmetic composition. The concentration of the additives is between 7.66 wt. % and 9.85 wt. %, and the additives are selected from the group consisting of 1,3-butanediol, hydroxyethyl cellulose, sodium glutamate, phenoxyethanol, ethylhexyl glycerol, and imidazolidinyl urea.

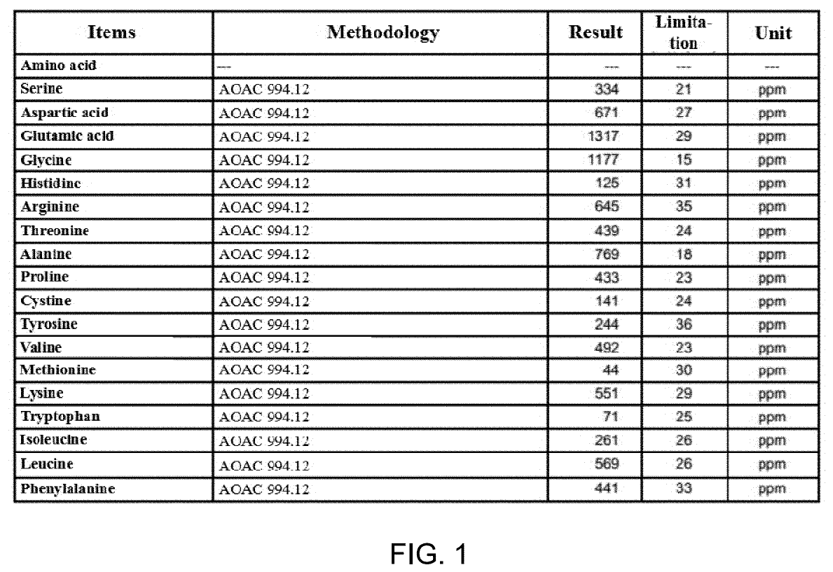

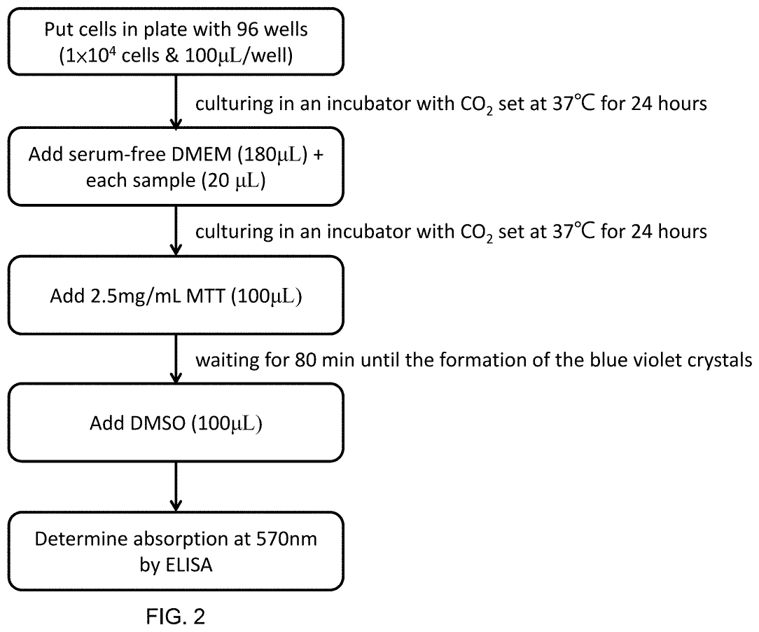

Chlorella Growth Factor (CGF) disclosed herein refers to extracted active ingredients which are originally contained in Chlorella. CGF may be extracted from the Chlorella through specific extraction methods, such as hot water extraction, and be capable of promoting the growth of cells. CGF may contain specific types of proteins, and the amino acid composition of proteins are listed in FIG. 1. Also, the physical characteristics of the CGF are listed in Table 1.

TABLE 1

| Characteristics | Results |
|---|---|
| Color | light brown |
| Taste | light and fresh grassy taste |
| pH | 6.2 |
| OD 260 nm (a.u.) | 440 |
| Chromaticity (a.u.) | 0.210 |
| Chloride (%) | 0.257 |
| Turbidity (a.u.) | 0.001 |
| Density | 1.022 |
| Total nitrogen (%) | 0.47 |
| Crude protein (%) | 2.91 |
| Total solid (%) | 3.73 |
| Total polyphenols (mg/100 ml)* | 49.3 |
| Superoxide dismutase (U/100 ml) | 675.39 |

*the amount of the total polyphenols is calculated based on the amount of the gallic acid

The term “OD 260 nm” listed in Table 1 refers to an optical density at a wavelength of 260 nm. Specifically, the term “OD 260 nm” is a base−10 log of the ratio “I₀/I”, where “I₀” represents the intensity of the incident light with a wavelength of 260 nm before the light passes through the solution or the substance, and “I” represents the intensity of the transmitted light after the light passes through the solution or the sub stance.

In addition, according to another embodiment of the invention, the cosmetic composition may further include γ-polyglutamate hydrogel, which has a cross-linked structure and a greater average molecular weight of between 15×10⁶ Da and 200×10⁶ Da.

According to the above embodiments, because the cosmetic compositions include γ-polyglutamic acid (γ-PGA) and chlorella growth factor (CGF), or further include γ-polyglutamate hydrogel, when a suitable amount of the cosmetic compositions is applied on the human skin for consecutive few days or weeks, the spots, wrinkles and textures on the skin may be removed effectively. Besides, the elasticity and collagen growth rate of the skin may also be promoted. In addition, chlorella growth factor may promote the growth of human keratinocytes (HaCaT) and 3T3 fibroblasts and improve the healing rate of 3T3 fibroblasts in vitro. Furthermore, the cosmetic compositions disclosed above do not show any irritating effect on the skin applied with the cosmetic compositions.

In order to have the person having ordinary skill in the art better understand the spirit and scope of the present invention, several embodiments are further disclosed in the following paragraphs.

<Cosmetic Composition>

The cosmetic compositions of control groups (items 1, 4, 5, 8-10, 12 and 13) and experimental groups (items 2, 3, 6, 7, and 11) are listed in Tables 2 and 3

TABLE 2

| | | | | | | Item | Item | | |
|---|---|---|---|---|---|---|---|---|---|
| Ingredients | Item 1 | Item 2 | Item 3 | Item 4 | Item 5 | 6(2D) | 7(2D&3D) | Item 8 | Item 9 |
| γ-PGA or its salts (wt. %) | — | — | — | — | — | 1~5 | 1~4 | — | 1~5 |
| CGF (wt. %) | — | — | — | — | — | 2~8 | 2~8 | 2~8 | — |
| γ-PGA hydrogel (wt. %) | — | 0.1 | 0.2 | — | — | — | 0.1~1 | — | — |
| Hyaluronic acid (HA) | — | — | — | 0.1 | 0.2 | — | — | — | — |
| (wt. %) | | | | | | | | | |
| Steareth-21 (wt. %) | 2 | 2 | 2 | 2 | 2 | — | — | — | — |
| Glyceryl Stearate (wt. %) | 4 | 4 | 4 | 4 | 4 | — | — | — | — |
| Stearyl Alcohol (wt. %) | 2 | 2 | 2 | 2 | 2 | — | — | — | — |
| Ethylhexyl | 8.5 | 8.5 | 8.5 | 8.5 | 8.5 | — | — | — | — |
| Stearate) (wt. %) | | | | | | | | | |
| Caprylic/Capric | 8.5 | 8.5 | 8.5 | 8.5 | 8.5 | — | — | — | — |
| Triglyceride (wt. %) | | | | | | | | | |
| Microcide BIP 3 (wt. %) | 0.1 | 0.1 | 0.1 | 0.1 | 0.1 | — | — | — | — |
| water (wt. %) | 74.9 | 74.8 | 74.7 | 74.8 | 74.7 | 77.15~89.34 | 77.15~89.24 | 82.15~90.34 | 82.15~90.34 |
| 1,3-Butylene Glycol | — | — | — | — | — | 6.0 | 6.0 | 6.0 | 6.0 |
| (wt. %) | | | | | | | | | |
| Hydroxyethyl Cellulose | — | — | — | — | — | 0.01-0.2 | 0.01-0.2 | 0.01-0.2 | 0.01-0.2 |
| (HEC) (wt. %) | | | | | | | | | |
| Monosodium glutamate | — | — | — | — | — | 1~3 | 1~3 | 1~3 | 1~3 |
| (MSG) (wt. %) | | | | | | | | | |
| Kem EHG | — | — | — | — | — | 0.25 | 0.25 | 0.25 | 0.25 |
| (phenoxyethanol + | | | | | | | | | |
| ethylhexyl glycerin) | | | | | | | | | |
| (wt. %) | | | | | | | | | |
| Imidazolidinyl urea | — | — | — | — | — | 0.4 | 0.4 | 0.4 | 0.4 |
| (wt. %) | | | | | | | | | |

TABLE 3

| Ingredients | Item 10 | Item 11 | Item 12 | Item 13 |
|---|---|---|---|---|
| Item 6(wt. %) | — | 1-10 | — | — |
| Item 8 [item 6 without γ-PGA or its salts] | — | — | 1-10 | — |
| (wt. %) | | | | |
| Item 9 [item 6 without CGF] (wt. %) | — | — | — | 1-10 |
| Glyceryl stearate (wt. %) | 3 | 3 | 3 | 3 |
| Water (wt. %) | 37.35 | 22.35-32.35 | 22.35-32.35 | 22.35-32.35 |
| 1,3-Butylene Glycol (wt. %) | 3 | 3 | 3 | 3 |
| Kem EHG (phenoxyethanol + Ethylhexyl | 0.25 | 0.25 | 0.25 | 0.25 |
| glycerol) (wt. %) | | | | |
| Imidazolidinyl urea (wt. %) | 0.4 | 0.4 | 0.4 | 0.4 |
| Glycerol monostearate (wt. %) | 2 | 2 | 2 | 2 |
| Myritol ® 318* (wt. %) | 3 | 3 | 3 | 3 |
| Isopropyl palmitate (wt. %) | 8 | 8 | 8 | 8 |
| Diethylhexyl carbonate (Tegosoft DEC) | 3 | 3 | 3 | 3 |
| (wt. %) | | | | |
| 1 wt. % Xanthan gum soln. (wt. %) | 40 | 40 | 40 | 40 |

*Myritol ® 318* is a product of BASF corp.

The effects of the cosmetic compositions listed in Tables 2 and 3 are subject to a series of tests, including <cell growth test>, <ex vivo moisture retaining ability test>, <moisture content and water loss of skin test>, <UV-induced damage test>, <cell wound healing test>, <skin irritation test>, <skin spots, wrinkles, texture test>, <skin elasticity, skin collagen content test>, <skin moisture content test>, <transepidermal water loss test>, which are disclosed in the following paragraphs.

<Cell Growth Test>

In the cell growth test, HaCaT cells and 3T3 fibroblasts were used to test the proliferative effect of the cosmetic compositions listed in Table 2 on the growth of cells. The test procedures were as follows: (1) culturing HaCaT/3T3 cells in 96 wells at 37° C., each well contains 1×10⁴ cells and 100 μL, of cytosol, the cells were cultured in an incubator set at 37° C. and 5% CO₂ for 24 hours; (2) removing the original culture medium from the wells, and adding 1804, of fresh culture medium (serum-free DMEM) for each well, and 20 μL, of test products and 20 μL, of control group were added with three replicates. The modified culture medium was placed in 37° C. incubator containing 5% CO₂ for 24 hours; (3) adding 1004, of 2.5 mg/mL MTT reagent, incubating in an incubator containing 5% CO₂ at 37° C. for 80 minutes; and (4) once blue-violet crystals were generated in the wells, the liquid in the wells was removed and mixed with 100 μL, DMSO; and (5) the liquid was then performed with enzyme-linked immunosorbent assay (ELISA) to measure its absorption at 570 nm. Cell survival rate was calculated according to the following formula (1):

$$cell survival rate ( \% ) = \frac{100 \times {OD}_{570 e}}{{OD}_{570 b}} \qquad ( 1 )$$

Where OD570e is the average optical density of the experimental groups, OD570b is the average optical density of the control group.

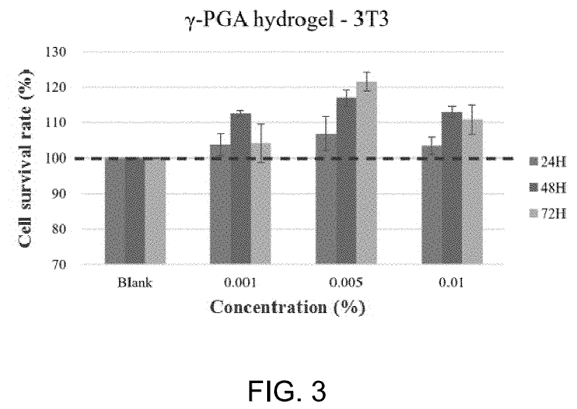

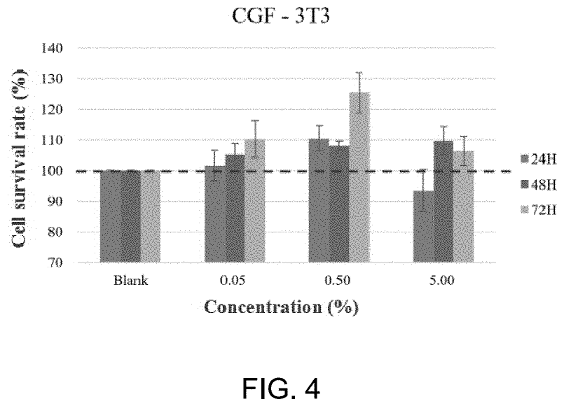

The test results were shown in FIG. 3 and FIG. 4. The growth rate of the 3T3 cells treated with the 0.005 wt. % γ-PGA hydrogel for 48 hr and 72 hr is obviously promoted (see FIG. 3). Besides, the growth rate of the 3T3 cells treated with the 0.5 wt. % CGF 72 hr is significantly induced. (see FIG. 4)

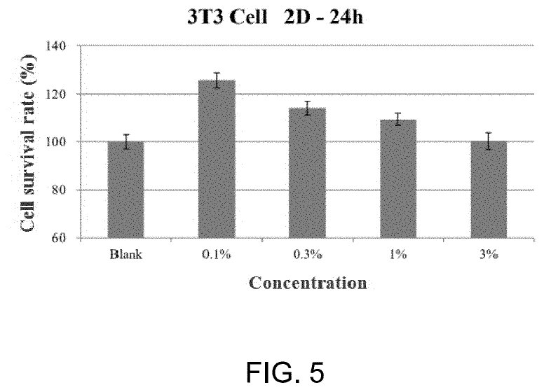

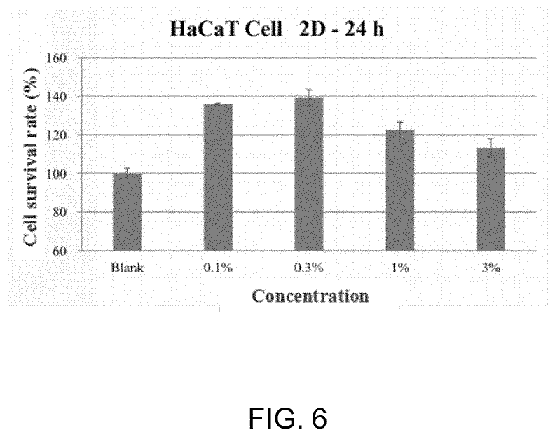

The cosmetic composition of item 6 (2D) was used as a test sample to test its growth-promoting effect on 3T3 cells. As shown in FIG. 5, 0.1 wt. % of item 6 has the best growth-promoting effect on 3T3 cells at 24 hours (125.57%). In addition, the cosmetic composition of item 6 (2D) was also used as a test sample to test its growth-promoting effect on HaCaT cells. The results were shown in FIG. 6. According to the results shown in FIG. 6, 0.3% item 6 (2D) has the best growth-promotion effect on HaCaT cells at 24 hours (139.25%).

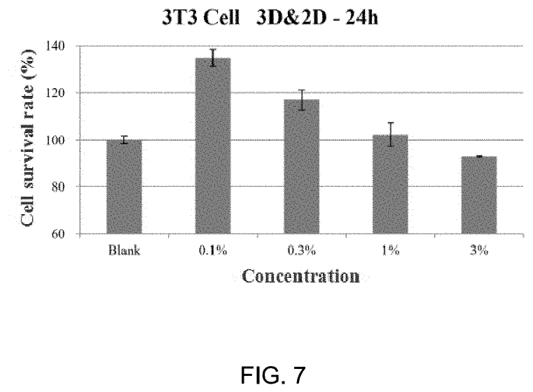

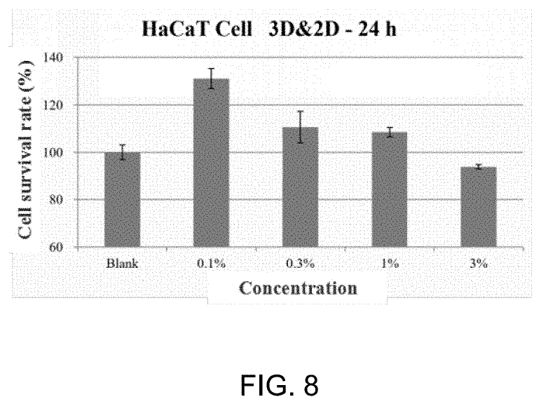

In addition, the cosmetic composition of item 7 (2D&3D) was used as a test sample to test its growth-promoting effect on 3T3 cells, as shown in FIG. 7. According to the results shown in FIG. 7, 0.1% item 7 (2D&3D) has the best growth-promoting effect on 3T3 cells at 24 hours (134.76%). In addition, the cosmetics composition of item 7 was used as a test sample to test its growth-promoting effect on HaCaT cells. The results were shown in FIG. 8. According to the results shown in FIG. 8, 0.1% item 7 (2D&3D) has the best growth-promotion effect on HaCaT cells at 24 hours (131.00%).

<Ex Vivo Moisture Retaining Ability Test>

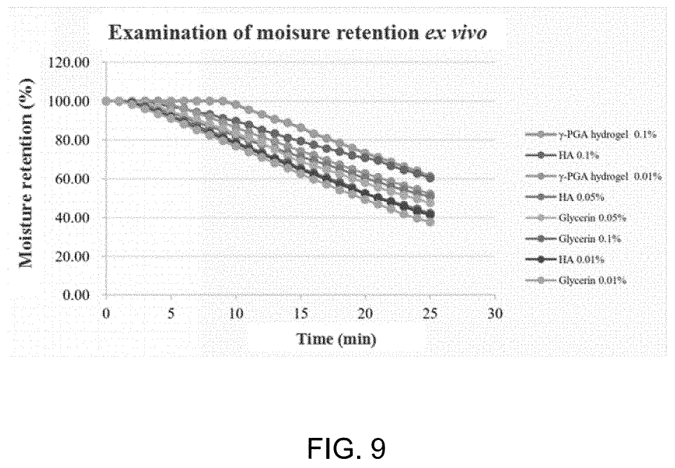

In this experiment, a clean filter paper was placed on the balance and reset to zero, and then 20 mg of mixture containing HA and γ-PGA hydrogel were added to the filter paper for 25 minutes, and the weight was recorded every minute to evaluate its moisture retaining ability. Various mixtures with different concentrations of HA and γ-PGA hydrogel were evaluated. The test results were shown in FIG. 9. According to the results shown in FIG. 9, during 25 minutes of external moisture retention test, 0.1 wt. % γ-PGA hydrogel (item 2) had the best ex vivo moisture retaining effect, which was superior to 0.1 wt. % HA (item 4). Also, 0.1 wt. % γ-PGA hydrogel (item 2) had a greater moisture retaining effect than glycerin.

Other test conditions are as follows: 8 participants, the time course was 0 hr, 0.5 hr, 1 hr, 2 hr and 3 hr respectively. The participants are healthy men and women aged 20-50, and the participants exclude pregnant women, patients with skin diseases, allergies, cancer and other chronic diseases, and the participants are not allowed to use other skin care products during the test.

<Test for Moisture Content and Water Loss of Skin>

The test instruments include: Courage+Khazaka electronic-MPA 5 (Corneometer® CM 825) to measure the moisture content of human skin, and Courage+Khazaka electronic-MPA 5 (Tewameter® TM 300) to measure the water loss of human skin. The skin on the inside of the arm was evaluated. The test procedure included the process of monitoring the skin moisture content and transepidermal water loss at the beginning of the test and 0.5, 1, 2, and 3 hours after applying the formulas described above to the skin. The participants must stay in the constant temperature and humidity environment (23±2° C., 55±5%) for 20 minutes before perform moisture content and water loss of skin assay.

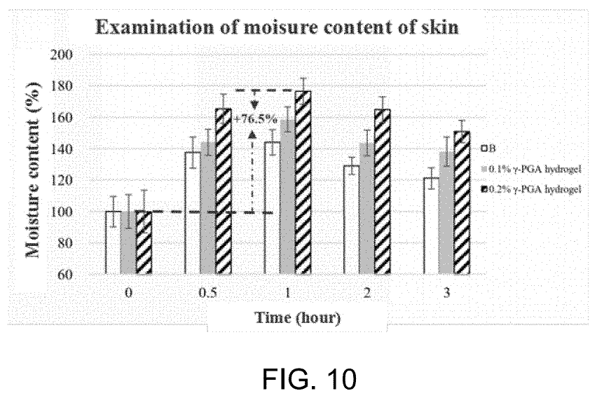

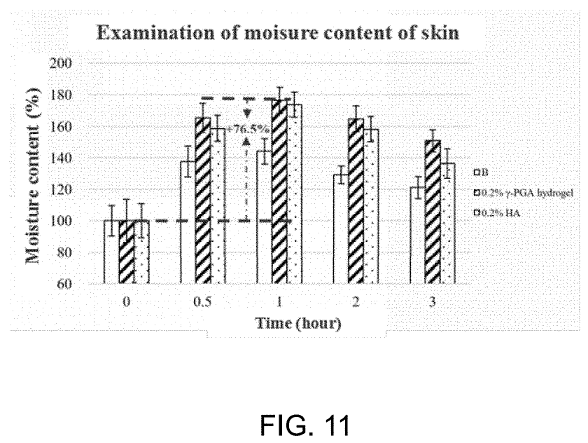

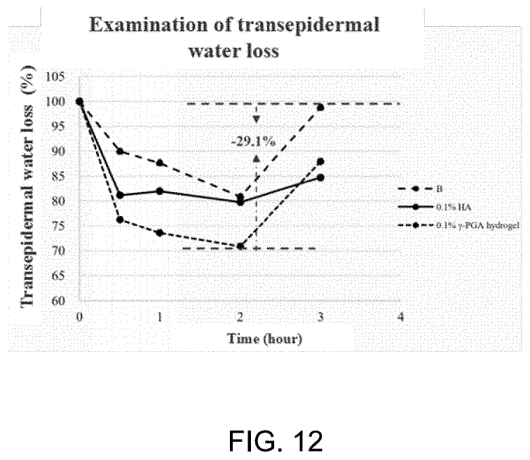

FIG. 10 shows skin moisture content with respect to different formulas and different time. According to the results shown in FIG. 10, compared with the skin moisture content (100%) at the beginning of the test, the skin moisture retaining ability was increased by 76.5% when 0.2 wt. % γ-PGA hydrogel (item 3) is applied for one hour, while the moisturizing effect may last for more than three hours. The data was expressed as Mean±SE, n=8. FIG. 11 shows skin moisture content with respect to different formulas and different time. Compared with the skin moisture content (100%) at the beginning of the test, the skin moisture was increased by 76.5% when 0.2 wt. % γ-PGA hydrogel (item 3) is applied for one hour, while the moisturizing effect may last for more than three hours. The moisture retaining ability of 0.2 wt. % γ-PGA hydrogel (item 3) is obviously better than 0.2 wt. % HA (item 5). The data was expressed as Mean±SE, n=8. FIG. 12 shows the result of transepidermal water loss with respect to different formulas and different time. Compared with the skin moisture content (100%) at the beginning of the test, the transepidermal water loss was decreased by 29.13% when 0.1 wt. % γ-PGA hydrogel (item 2) was applied for two hours, which was better than the transepidermal water loss with respect to 0.1 wt. % HA (item 4). The data was expressed as Mean±SE, n=8.

<UV-Induced Damage Test>

In the UV-induced damage test, 3T3 fibroblasts were used as test cells to test the protective effect of the cosmetic compositions listed in Table 2 on the cells. The test procedures were as follows: (1) culturing 3T3 cells in 96 wells at 37° C., each well contains 2×10⁴ cells and 100 μL, of cytosol, the cells were cultured in an incubator set at 37° C. and 5% CO₂ for 24 hours; (2) removing the original culture medium from the wells, and adding 180 μL, fresh culture medium (including 1×NEAA), and 20 μL, of test products and 20 μL, of control group for three replicates. The modified culture medium was placed in 37° C. incubator containing 5% CO₂ for 24 hours; and (3) adding 204, of MTT reagent, incubating in an incubator containing 5% CO₂ at 37° C. for 3 hours. Once blue violet crystals are generated in the wells, the liquid in the wells is removed and mixed with 100 μL, DMSO. After mixing it well, then ELISA was performed and measured the absorbance at 570 nm. Cell survival rate was calculated according to the following formula (1):

$$cell survival rate ( \% ) = \frac{100 \times {OD}_{570 e}}{{OD}_{570 b}} \qquad ( 1 )$$

Where OD570e is the average light absorption of the test samples, OD570b is the average light absorption of the control group (without UV exposure).

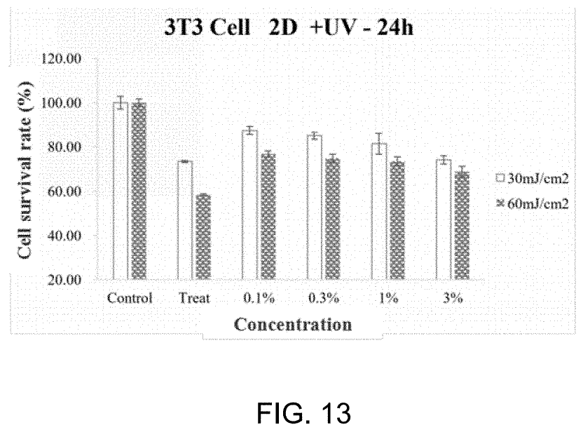

As shown in FIG. 13, compared with UV-treated sample (73.38%) receiving 24 hr UV (30mJ/cm²) exposure, the protective effect is 14.07% for test sample with 0.1 wt. % item 6 (2D) (87.45%) receiving 24 hr UV (30mJ/cm²) exposure. Similarly, compared with UV-treated sample (58.43%) receiving 24 hr UV (60mJ/cm²) exposure, the protective effect is increased to 18.36% for test sample with 0.1 wt. % item 6 (2D) (76.79%) receiving 24 hr UV (60mJ/cm²) exposure. The term “control” shown in FIG. 13 represents samples without exposing to any UV light, and the cell survival rate for these samples are 100%.

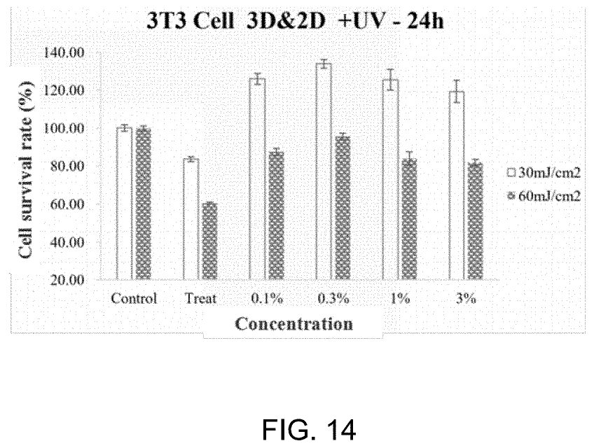

As shown in FIG. 14, compared with UV-treated sample (83.63%) receiving 24 hr UV (30mJ/cm²) exposure, the protective effect is 50.36% for test sample with 0.3 wt. % item 7 (2D&3D) (133.99%) receiving 24 hr UV (30mJ/cm²) exposure. Similarly, compared with UV-treated sample (60.45%) receiving 24 hr UV (60mJ/cm²) exposure, the protective effect is 35.08% for test sample with 0.3 wt. % item 7 (2D&3D) (95.53%) receiving 24 hr UV (60mJ/cm²) exposure. The term “control” shown in FIG. 14 represents samples without exposing to any UV light, and the cell survival rate for these samples are 100%.

<Cell Wound Healing Test>

In the cell wound healing test, 3T3 fibroblasts were used as test cells. The test procedures were as follows: (1) culturing 3T3 cells in 24 wells, each well contains 6×10⁴ cells and 800 μL, of cytosol, the cells were cultured in an incubator set at 37° C. and 5% CO₂ for 24 hours; (2) removing the original culture medium from the wells after 30 minutes, and then adding 8004, DMEM culture medium containing 1% CCS to the well. After 30 minutes, a 200 μL, tip was used to create the intercellular space, and the DMEM medium containing 1% CCS was removed. Then, 780 μL, DMEM medium containing 1% CCS and 20 μL, of test samples and 20 μL, of control samples were added and cultured in an incubator containing 5% CO₂ at 37° C.; (3) observing and photographing by a microscope at 0 hr and 24 hr after the addition of samples; (4) the cells were fixed with 95% ethanol (containing 5% glacial acetic acid) at 24 hr and stained with 0.2% methyl blue 5 minutes after the addition of the 95% ethanol. The healing rate was defined as follows:

$$healing rate = ( 1 - \frac{area of blank region at 0 hr}{area of blank region at 24 hr} ) \times 100$$

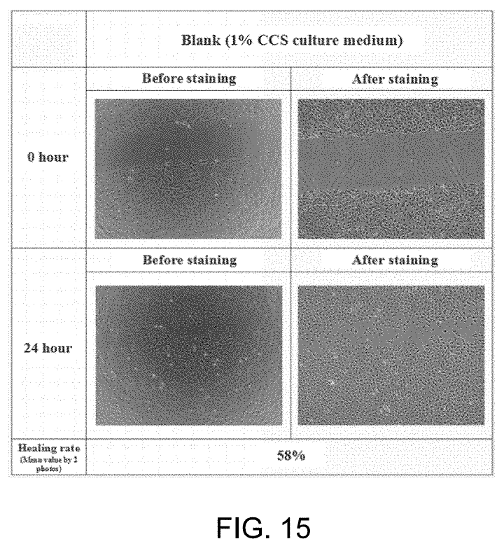

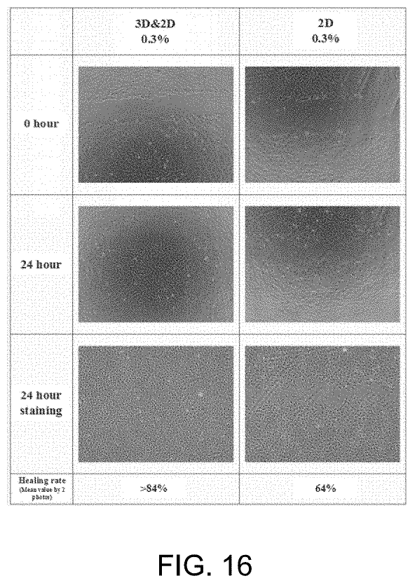

FIG. 15 shows the test result at 0 hr and 24 hr during the test for control samples (item 1, without active ingredients). The healing rate is only 58%. In contrast, FIG. 16 shows the test results at 0 hr and 24 hr during the test for test samples (items 6 and 7), which showed that item 6 (2D) effectively promoted healing effect, and healing rate is up to 64%. Item 7 (2D&3D) had the best healing effect, and the healing rate was up to 84%.

<Skin Irritation Test>

In the skin irritation test, the test procedures include: (1) making the participants wait in an environment of constant temperature and humidity (23±2° C., 55±5%) for 20 minutes; (2) conducting a skin patch test based on ISO-10993-10, which includes defining 4 blocks with 2×2 cm², such as blocks A, B, C, and D, on the right and left arms of each participant. Specifically, block A is on the right arm and applied with non-diluted item 7 (3D&2D), block B is on the right arm and applied with diluted item 7 (3D&2D, 30 wt. %), block C is on the left arm and applied with non-diluted item 6 (2D), block D is on the left arm and applied with diluted item 6 (2D, 30 wt. %). The patch is removed from the arms 24 hr after the ingredients are applied to the blocks on the skin. The tested skin was observed by naked eyes 24 hr and 48 hr after the ingredients are applied to the blocks A-D on the skin. The tested skin was evaluated based on modified Draize evaluation method (Table 4) to obtain primary irritation index (PII), and determine the classification of skin irritation reaction (Table 5); (3) evaluating the irritation of the product to human skin, which includes applying the products to the inside of the arms and evaluating the tested skin by Mexameter® MX18 (The Multi-ProbeAdapter System® MPA-5, Courage+Khazaka, Germany) at 0 hr, 24 hr, 48 hr after applying the products on the skin. The skin condition is recorded; (4) representing corresponding statistical results based on mean±standard error (SE).

TABLE 4

| Skin response to irritation | Skin irritation value |
|---|---|
| The formation of erythema | |
| and scab - up to 4 points | |
| No erythema | 0 |
| Just discernible slight erythema | 1 |
| Obvious erythema | 2 |
| Moderate to strong erythema | 3 |
| Very red (e.g. beetroot) erythema | 4 |
| to mild scab formation | |
| (bleeding, ulcers, and tissue | |
| necrosis also fall into | |
| this category) | |
| Formation of swelling - up to 4 points | |
| No swelling | 0 |
| Just discernible slight edema | 1 |
| Slightly visible edema compared with | 2 |
| surrounding area | |
| Moderate edema (protruding about 1 mm) | 3 |
| Severe swelling (protruding over 1 mm) | 4 |
| and spread to the skin beyond exposure | |

TABLE 5

| Skin irritation index (PII) | Classification |
|---|---|
| 0.0-0.4 | Non-irritating |
| 0.5-1.9 | Slightly irritating |
| 2.0-4.9 | Moderately irritating |
| 5.0-8.0 | Severely irritating |

For the skin condition, no skin erythema, swelling and inflammation were observed before and after the 48 hr patch test. According to the evaluation method of modified Draize, the score of irritation response is 0.0 for items 6 and 7 (non-diluted or diluted), which indicates no skin irritation.

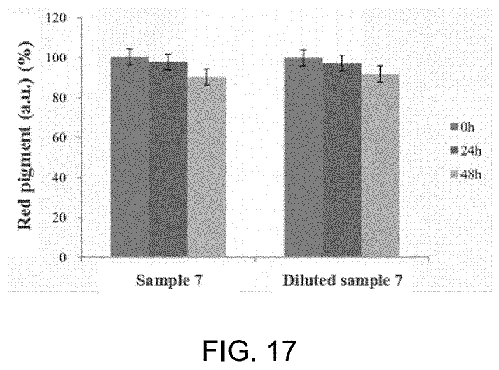

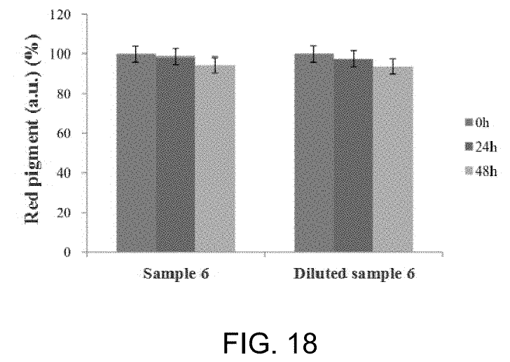

In addition, referring to FIGS. 17 and 18, the results evaluated by the Mexameter® MX18 showed that there is no significant difference in the amount of the skin erythema for items 6 and 7 (non-diluted and diluted) at 0 hr, 24 hr and 48 hr after the use of the products. Besides, the number of the skin erythema does not increase or even slightly decreases, which means that items 6 and 7 may not irritate the skin.

<Skin Spots, Wrinkles and Texture Test>, <Skin Elasticity, Skin Collagen Content Test>, <Skin Moisture Content Test>, and <Transepidermal Water Loss Test>

Exemplary Cosmetic Compositions Regarding Items 6, 7 and 11

The instruments used in the test include Canfield VISTA Complexion Analysis System (skin spots, wrinkles, texture are identified through detecting and comparing skin images), DermaLab® (suction cup units) (measuring skin elasticity, skin collagen content test), Courage+Khazaka electronic-MPA 5 (Corneometer® CM 825)(human skin moisture content), Khazaka electronic-MPA 5 (Tewameter® TM 300) (human skin moisture content and water loss). The tested portions include the forehead and both sides of the cheek (VISIA Complexion Analysis System), outer corner of the eyes (DermaLab® (suction cup units)), both sides of the cheek (German, Courage+Khazaka-MPA 5 (Corneometer® CM 825)), both sides of the cheek (German, Courage+Khazaka electronic—MPA 5 (Tewameter® TM 300)). The detailed test procedures are as follows: wait for 10 min to measure VISTA (spots, wrinkles, texture and pores), and wait for 20 min to test skin elasticity, skin collagen content, skin moisture content and transepidermal water loss. The tested skin was measured at day 0 (before use), day 7, day 14, day 28, day 42 and day 56 after use. Statistical data were expressed as mean±standard error (SE), and analyzed by analysis of variance (ANOVA) and T-test. In a condition where *p<0.05, **p<0.01, ***p<0.001, there was a significant statistical difference between the two experimental groups.

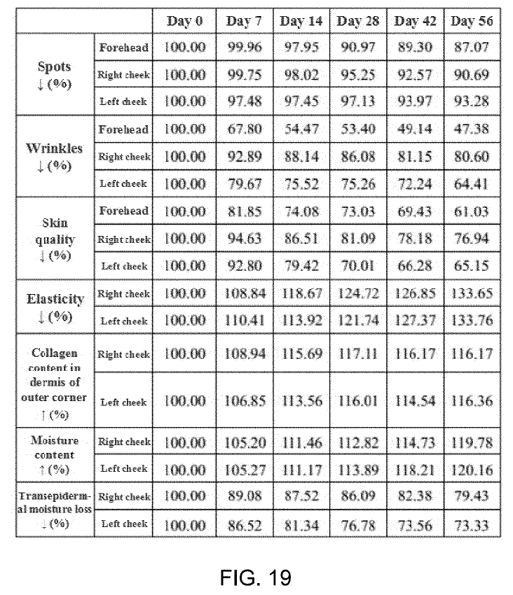

Other test conditions were as follows: there were a total of 20 female participants with healthy skin and aged between 25 and 60. Test results were shown in FIG. 19.

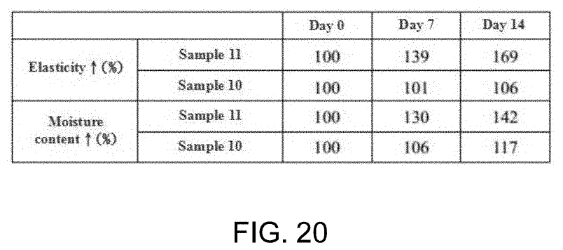

According to the results shown in FIG. 19, skin spots were significantly improved after using the product for 56 days compared with the skin spots at the beginning of the test. The spots in the forehead were improved by 12.93%. Also, skin wrinkles were improved significantly after using the product for 7 days compared with the skin wrinkles at the beginning of the test. Especially, the wrinkles in the forehead and the left cheek were respectively improved by 32.20% and 20.33%. The improvement in the wrinkles lasted to 56 days (52.62% for the forehead and 35.59% for the left cheek). Skin texture was also improved significantly after 14 days of use, with a 20.58% reduction on the left cheek. The improvement in the skin texture lasted to 56 days (34.85%). Skin elasticity was increased by 33.65% on the right cheek and 33.76% on the left cheek after 56 days of use. Skin collagen content was increased by 16.17% on the right cheek and 16.36% on the left cheek after 56 days of use. After 14 days of use, skin moisture content was increased by 11.46% on the right cheek and 11.17% on the left cheek, and by 19.78% on the right cheek and 20.16% on the left cheek after 56 days of use. According to the experimental results, the cosmetic compositions in Table 2 (items 6 and 7) can rapidly reduce wrinkles within 7 days, improve skin texture at 14 days, increase skin collagen production, skin elasticity, skin moisture content, and reduce transepidermal water loss. The continuous use of the cosmetic compositions in Table 2 (items 6 and 7) not only continuously improves skin spots, wrinkles, and textures, but also continuously promotes skin elasticity, skin collagen production, and continuously increases skin moisture content and reduces transepidermal water loss. In addition, according to the test results shown in FIG. 20, the continuous use of the cosmetic compositions in Table 3 (item 11 in emulsion form) may also increase skin elasticity and skin moisture content.

Cosmetic Compositions Regarding Comparative Examples (Items 10, 12 and 13) and an Exemplary Example (Item 11)

The instruments used in the test include Canfield VISIA Complexion Analysis System (skin spots, wrinkles, texture are identified through detecting and comparing skin images), DermaLab Ultrasound (skin collagen firmness test), DermaLab® (suction cup units) (skin elasticity test). The tested portions include the forehead and both sides of the cheek (VISTA Complexion Analysis System), outer corner of the eyes (DermaLab Ultrasound, DermaLab® (suction cup units)). The detailed test procedures are as follows: participants are applied with the samples in the morning and the evening for consecutive 56 days. Two different samples (0.6 g for each) were respectively applied to the right forehead (or left forehead) and the right cheek (or left cheek) of the participant. The samples were not applied around the eyes, and did not need to be removed after use. The tested skin was measured at day 0 (before use), day 7, day 14, day 28 and day 56 after use. Statistical data were expressed as mean±standard error (SE), and analyzed by analysis of variance (ANOVA) and T-test. In a condition where *p<0.05, **p<0.01, ***p<0.001, there was a significant statistical difference between the two experimental groups.

Other test conditions were as follows: a total of 18 participants (17 females and 1 male) with healthy skin aged between 20 and 60 years old.

The results of <skin wrinkle test>, <skin collagen content test>, and <skin elasticity test> are described below.

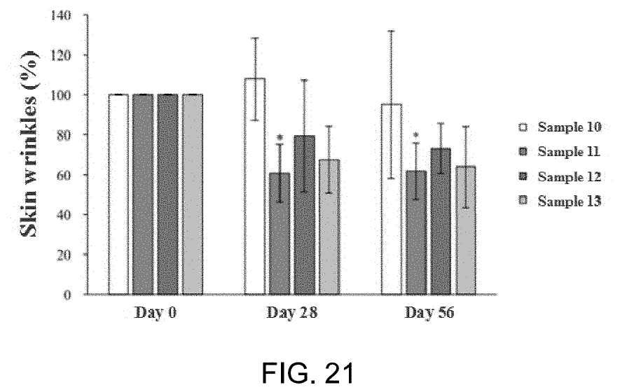

For the results of <skin wrinkles test>, referring to FIG. 21, after 28 days of use of the product, the wrinkles of the participants using item 11 were decreased effectively, and the improvement in skin wrinkles remained at a certain level after 56 days of consecutive use. In contrast, the improvement in skin wrinkles with respect to items 12 and 13 was significantly lower than that of item 11.

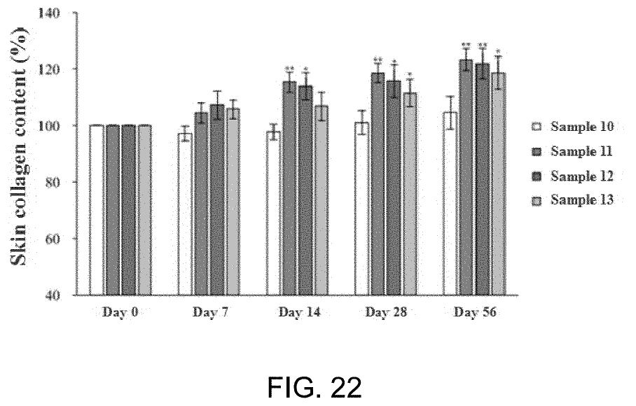

For the results of <skin collagen content test>, referring to FIG. 22, for the participants using the item 11, their collagen content was 115.35% after 14 days of use, which was 15% greater than the collagen content at the beginning of the test. Besides, collagen content was up to 123.3% after 56 days of use, which was 23% greater than the collagen content at the beginning of the test. In contrast, there was little change in collagen contents among participants using item 10. In addition, for the participants using item 12 and 13, the collagen content increased only by 21% and 18% respectively after 56 days of use, and the increase in the collagen content was lower than that of item 11.

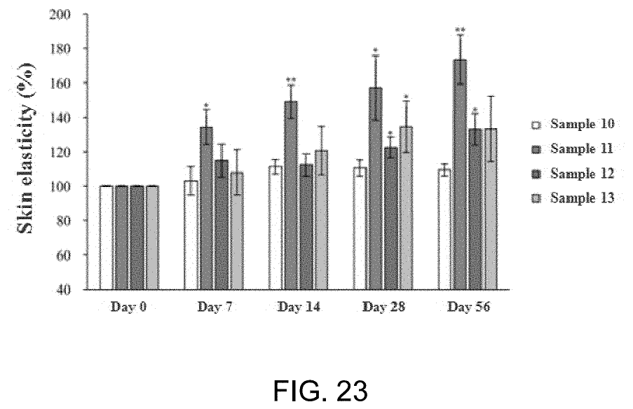

For the results of <skin elasticity test> were shown in FIG. 23. For the participants using the item 11, their skin elasticity was 149.14% after 14 days of use, which was 49% greater than the skin elasticity at the beginning of the test. Besides, the skin elasticity was up to 173.52% after 56 days of use, which was 73% greater than the skin elasticity at the beginning of the test. In contrast, there was little change in skin elasticity among participants using item 10. In addition, for the participants using item 12 and 13, the skin elasticity increased only by 22% and 34% respectively after 28 days of use, and the increase in the skin elasticity was obviously lower than that of item 11.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A cosmetic composition comprising γ-polyglutamic acid or a salt thereof as active ingredients, comprising:
from 0.01% to 5% by weight of γ-polyglutamic acid (γ-PGA) or a salt thereof, wherein an average molecular weight of the γ-polyglutamic acid or the salt thereof is between 1×10⁶ Da and 3×10⁶ Da based on a total weight of the cosmetic composition; and
from 0.02% to 8% by weight of chlorella growth factor (CGF) based on a total weight of the cosmetic composition.

2. The cosmetic composition of claim 1, further comprising from 7.66% to 9.85% by weight of additives based on a total weight of the cosmetic composition.

3. The cosmetic composition of claim 2, wherein the additives are selected from the group consisting of 1,3-butanediol, hydroxyethyl cellulose, sodium glutamate, phenoxyethanol, ethylhexyl glycerol, and imidazolidinyl urea.

4. The cosmetic composition of claim 1, further comprising a γ-polyglutamate hydrogel.

5. The cosmetic composition of claim 4, wherein an average molecular weight of the γ-polyglutamate hydrogel is between 15×10⁶ Da and 200×10⁶ Da.

6. The cosmetic composition of claim 4, wherein the γ-polyglutamate hydrogel comprises a cross-linked structure.

7. The cosmetic composition of claim 1, wherein the cosmetic composition is capable of reducing skin wrinkles of a user.

8. The cosmetic composition of claim 1, wherein the cosmetic composition is capable of increasing a collagen content of a user's skin.

9. The cosmetic composition of claim 1, wherein the cosmetic composition is capable of increasing skin elasticity of a user.
